SEQUENCE LISTING (56 sequences)

SEQ ID NO: 1 - protein, 21 aa, Mus musculus
MEVGWYRSPFSRVVHLYRNGK

SEQ ID NO: 2 - protein, 21 aa, Homo sapiens
MEVGWYRPPFSRVVHLYRNGK

SEQ ID NO: 3 - protein, 9 aa, Homo sapiens
SLLLELEEV

SEQ ID NO: 4 - protein, 9 aa, Mus musculus
SLYLDLEEV

SEQ ID NO: 5 - protein, 9 aa, Unknown
  source: /note=“Description of Unknown Mouse or human MAG509-517 sequence”
LMWAKIGPV

SEQ ID NO: 6 - protein, 9 aa, Homo sapiens
VLFSSDFRI

SEQ ID NO: 7 - protein, 9 aa, Mus musculus
VLYSPEFRI

SEQ ID NO: 8 - protein, 9 aa, Homo sapiens
SLSRFSWGA

SEQ ID NO: 9 - protein, 9 aa, Mus musculus
SLSRFSWGG

SEQ ID NO: 10 - protein, 9 aa, Unknown
  source: /note=“Description of Unknown mouse or human MOG sequence”
KVEDPFYWV

SEQ ID NO: 11 - protein, 9 aa, Unknown
  source: /note=“Description of Unknown mouse or human MOG sequence”
RTFDPHFLR

SEQ ID NO: 12 - protein, 9 aa, Unknown
  source: /note=“Description of Unknown mouse or human MOG sequence”
FLRVPCWKI

SEQ ID NO: 13 - protein, 10 aa, Unknown
  source: /note=“Description of Unknown mouse or human MOG sequence”
KITLFVIVPV

SEQ ID NO: 14 - protein, 9 aa, Unknown
  source: /note=“Description of Unknown mouse or human MOG sequence”
VLGPLVALI

SEQ ID NO: 15 - protein, 9 aa, Unknown
  source: /note=“Description of Unknown mouse or human MOG sequence”
TLFVIVPVL

SEQ ID NO: 16 - protein, 10 aa, Unknown
  source: /note=“Description of Unknown mouse or human MOG sequence”
RLAGQFLEEL

SEQ ID NO: 17 - protein, 9 aa, Unknown
  source: /note=“Description of Unknown mouse or human MOG sequence”
FLYGALLLA

SEQ ID NO: 18 - protein, 14 aa, Unknown
  source: /note=“Description of Unknown MOG sequence”
HPIRALVGDEVELP

SEQ ID NO: 19 - protein, 20 aa, Unknown
  source: /note=“Description of Unknown MOG sequence”
VGWYRPPFSRVVHLYRNGKD

SEQ ID NO: 20 - protein, 26 aa, Unknown
  source: /note=“Description of Unknown MOG sequence”
LKVEDPFYWVSPGVLVLLAVLPVLLL

SEQ ID NO: 21 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 22 - protein, 11 aa, Homo sapiens
MRLLPLLALLA

SEQ ID NO: 23 - protein, 15 aa, Homo sapiens
SHLVEALYLVCGERG

SEQ ID NO: 24 - protein, 9 aa, Homo sapiens
LYLVCGERG

SEQ ID NO: 25 - protein, 15 aa, Homo sapiens
GIVEQCCTSICSLYQ

SEQ ID NO: 26 - protein, 5 aa, Homo sapiens
  VARIANT (1)..(1): /replace=“Arg”
  VARIANT (2)..(2): /replace=“Arg”
  SITE (1)..(5): /note=“Variant residues given in the sequence have no preference with respect to those in the annotations for variant positions”
QKRAA

SEQ ID NO: 27 - protein, 8 aa, Homo sapiens
AGERGPPG

SEQ ID NO: 28 - protein, 15 aa, Homo sapiens
AGGFDEKAGGAQLGV

SEQ ID NO: 29 - protein, 11 aa, Homo sapiens
VGPAGGPGFPG

SEQ ID NO: 30 - protein, 30 aa, Homo sapiens
SLKLMATLFSTYASADTGDSGKGKGGKKKG

SEQ ID NO: 31 - protein, 14 aa, Homo sapiens
GQFIDSGKAGAEKL

SEQ ID NO: 32 - protein, 14 aa, Homo sapiens
DECSELKKDIDDLE

SEQ ID NO: 33 - protein, 12 aa, Homo sapiens
  MOD_RES (5)..(5): 3,5,3′,5′-tetraiodothyronine
NIFEXQVDAQPL

SEQ ID NO: 34 - protein, 22 aa, Homo sapiens
  MOD_RES (10)..(10): 3,5,3′,5′-tetraiodothyronine
YSLEHSTDDXASFSRALENATR

SEQ ID NO: 35 - protein, 20 aa, Homo sapiens
  MOD_RES (10)..(10): 3,5,3′,5′-tetraiodothyronine
RALENATRDXFIICPIIDMA

SEQ ID NO: 36 - protein, 14 aa, Homo sapiens
  MOD_RES (12)..(12): 3,5,3′,5′-tetraiodothyronine
LLSLQEPGSKTXSK

SEQ ID NO: 37 - protein, 16 aa, Homo sapiens
  MOD_RES (7)..(7): 3,5,3′,5′-tetraiodothyronine
EHSTDDXASFSRALEN

SEQ ID NO: 38 - protein, 14 aa, Homo sapiens
LKKRGILSPAQLLS

SEQ ID NO: 39 - protein, 16 aa, Homo sapiens
SGVIARAAEIMETSIQ

SEQ ID NO: 40 - protein, 14 aa, Homo sapiens
PPVREVTRHVIQVS

SEQ ID NO: 41 - protein, 15 aa, Homo sapiens
PRQQMNGLTSFLDAS

SEQ ID NO: 42 - protein, 15 aa, Homo sapiens
LTALHTLWLREHNRL

SEQ ID NO: 43 - protein, 15 aa, Homo sapiens
HNRLAAALKALNAHW

SEQ ID NO: 44 - protein, 14 aa, Homo sapiens
ARKVVGALHQIITL

SEQ ID NO: 45 - protein, 16 aa, Homo sapiens
LPGLWLHQAFFSPWTL

SEQ ID NO: 46 - protein, 17 aa, Homo sapiens
MNEELTERLFVLSNSST

SEQ ID NO: 47 - protein, 11 aa, Homo sapiens
LDLASINLQRG

SEQ ID NO: 48 - protein, 16 aa, Homo sapiens
RSVADKILDLYKHPDN

SEQ ID NO: 49 - protein, 14 aa, Homo sapiens
IDVWLGGLAENFLP

SEQ ID NO: 50 - protein, 12 aa, Homo sapiens
QQQHERRKQERK

SEQ ID NO: 51 - protein, 14 aa, Homo sapiens
PTKEIEIQVDWNSE

SEQ ID NO: 52 - protein, 6 aa, Artificial Sequence
  source: /note=“Description of Artificial Sequence Synthetic 6xHis tag”
HHHHHH

SEQ ID NO: 53 - protein, 12 aa, Artificial Sequence
  source: /note=“Description of Artificial Sequence Synthetic peptide”
VYFCAVRSYNQG

SEQ ID NO: 54 - protein, 11 aa, Artificial Sequence
  source: /note=“Description of Artificial Sequence Synthetic peptide”
CASSLDPGANT

SEQ ID NO: 55 - protein, 12 aa, Artificial Sequence
  source: /note=“Description of Artificial Sequence Synthetic peptide”
VYFCALRSYNFG

SEQ ID NO: 56 - protein, 11 aa, Artificial Sequence
  source: /note=“Description of Artificial Sequence Synthetic peptide”
CASSLDCGANP

(19) United States
(12) Patent Application Publication
(10) Pub. No.: US 2021/0061875 A1
(43) Pub. Date: Mar. 4, 2021

(54) COMPOSITIONS AND METHODS FOR TREATING AUTOIMMUNE DISEASE

(71) Applicant: The Henry M. Jackson Foundation for the Advancement of Military Medicine, Inc., Bethesda, MD (US)

(72) Inventor: Jason R. LEES, Ellicott City, MD (US)

(73) Assignee: The Henry M. Jackson Foundation for the Advancement of Military Medicine, Inc., Bethesda, MD (US)

(21) Appl. No.: 16/958,539

(22) PCT Filed: Dec. 28, 2018
(86) PCT No.: PCT/US2018/067832

§ 371 Date: Jun. 26, 2020

Related U.S. Application Data
(60) Provisional application No. 62/612,096, filed on Dec. 29, 2017.

(51) Int. Cl. C07K 14/725; C07K 14/705; A61K 35/17; A61K 45/06; C12N 5/0783; A61P 37/06; C07K 16/28

(52) U.S. Cl. CPC C07K 14/7051; C07K 14/70521; C07K 14/70578; A61K 35/17; A61K 45/06; C12N 2510/00; A61P 37/06; C07K 16/2803; C07K 2319/03; C07K 2319/33; C07K 2317/622; C12N 5/0636

(57) ABSTRACT

Provided herein are compositions comprising engineered CD8+ T cells that express a heterologous T cell receptor (TCR) having specificity for an autoantigen bound to a Major Histocompatibility Complex (MHC) Class II. Also provided are methods for the treatment of an autoimmune disease comprising administering the engineered CD8+ T cells. Also provided are methods for generating engineered CD8+ T cells that express a heterologous MHC Class II TCR, including methods of isolating autoantigen-MHC class II specific TCR for use in engineering CD8+ T cells for treatment.

### CROSS-REFERENCE TO RELATED APPLICATIONS

This application claims priority to U.S. provisional application 62/612,096, filed Dec. 29, 2017, the entire contents of which are incorporated herein by reference.

### BACKGROUND

T cell recognition of cognate antigen is a crucial factor driving both activation steps and initiation of T cell effector function, a process crucial to autoimmune induction and pathogenesis. CD4+ T cells typically only recognize antigenic peptides in the context of major histocompatibility (MHC) class II molecules, and the expression patterns of MHC class II are limited, thus restricting CD4+ T cell effector activation to interactions with MHC class II expressing antigen presenting cells (APC). Importantly, many of the cells targeted in tissue-specific autoimmunity do not appear to express MHC class II molecules and so cannot be directly targeted by CD4 cells.

In multiple sclerosis (MS), CD4+ T cells are thought to coordinate initiation and maintain neuroinflammation following recognition of central nervous system (CNS) antigens in the context of MHC class II molecules expressed on the surface of APC. Recognition of CNS antigens at the CNS is required to drive CD4+ T cell re-activation and subsequent production of cytokines and chemokines crucial to the recruitment of secondary immune effector cells. These CD4+ T cell effector functions in turn result in induction of immune pathology while also priming APC to enhance future T cell-APC interactions. MHC class II, however, is not expressed on oligodendrocytes and neurons, two cellular targets for MS pathology.

Type 1 diabetes is an organ-specific autoimmune disease caused by the autoimmune response against pancreatic β cells. Type 1 diabetes is often complicated with other autoimmune diseases, and anti-islet autoantibodies may precede the clinical onset of disease. While groups have reported production of MHC class II molecules in the pancreatic islet cells targeted in type I diabetes, functional surface expression of these molecules on the islet cell has not been shown.

Recent studies in cancer immunotherapy have defined protocols for inducing expression of chimeric antigen-specific T cell receptors (CAR) in mature CD8+ T cells for the purpose of inducing destruction of antigen bearing cells, apparently without modulating the underlying effector profile of the transduced T cell (i.e., CD8 cells are still cytotoxic through perforin and granzymes).

There remains a need for compositions and methods for treating autoimmune diseases, including MS and Type 1 diabetes.

### SUMMARY

Described herein are CD8+ T cells engineered to express a heterologous T cell receptor specific for an auto-antigen (self-antigen) bound to MHC class II, and methods for using the same, such as in adoptive cell therapy.

Described herein, in certain embodiments, are engineered CD8+ T cells comprising a heterologous nucleic acid encoding a T cell receptor (TCR), wherein the TCR binds to a self-antigen bound to a major histocompatibility complex (MHC) class II. In some embodiments, the engineered CD8+ T cell binds to a cell that expresses the self-antigen bound to MHC class II. In some embodiments, the cell that expresses the self-antigen bound to MHC class II is a dendritic cell, a macrophage, a monocyte, a microglial cell, or an astrocyte. In some embodiments, the MHC class II comprises H-2A, HLA-DP, HLA-DM, HLA-DOA, HLA-DOB, HLA-DQ, or HLA-DR. In some embodiments, the MHC class II comprises HLA-DR2, HLA-DR3, HLA-DR4, HLA-DR11, HLA-DR15 or HLA-DQ6.

In accordance with any of the foregoing embodiments, the engineered CD8+ T cell may be able to lyse a cell that expresses the self-antigen bound to MHC class II. In accordance with any of the foregoing embodiments, the engineered CD8+ T cell may decrease activation of CD4+ cells when administered to a subject having an autoimmune disease. In some embodiments, the autoimmune disease is a neuroinflammatory disease. In some embodiments, the autoimmune disease is multiple sclerosis, Type I diabetes, rheumatoid arthritis, myasthenia gravis, psoriasis, systemic lupus erythematosus, autoimmune thyroiditis, Graves' disease, inflammatory bowel disease, autoimmune uveoretinitis, myocarditis, and polymyositis.

In some embodiments, the self-antigen is a central nervous system (CNS) antigen. In some embodiments, such as embodiments relating to multiple sclerosis, the self-antigen is selected from myelin oligodendrocyte glycoprotein (MOG), myelin basic protein (MBP), myelin associated glycoprotein (MAG), and proteolipid protein (PLP). In some embodiments, such as embodiment relating to multiple sclerosis, the self-antigen is MOG35-55. In some embodiments, such as embodiments relating to Type I diabetes, the self-antigen is a diabetes mellitus-associated antigen, such as insulin, chromogranin A, glutamic acid decarboxylase 1 (GAD67), glutamic acid decarboxylase 2 (GAD65) or islet-specific glucose-6-phosphatase catalytic subunit-related protein. In some embodiments, such as embodiments related to other autoimmune diseases, the self-antigen is a rheumatoid arthritis associated antigen, myocarditis associated self-antigen, or a thyroiditis associated antigen.

In some embodiments, the nucleic acid encoding the TCR is operably linked to an inducible promoter or a conditional promoter.

Also described herein are engineered CD8+ T cells comprising a heterologous nucleic acid encoding a T cell receptor (TCR), wherein the TCR is derived from a CD4+ T cell. In some embodiments, the TCR is 2D2, B8, or bdc2.5. In some embodiments, the TCR is a chimeric antigen receptor (CAR) comprising (i) an extracellular antigen binding domain; (ii) a transmembrane domain; and (iii) an intracellular domain. In some embodiments, the extracellular antigen binding domain binds to the self-antigen bound to MEW Class II. In some embodiments, the extracellular antigen binding domain is derived from an antigen-binding portion of an antibody, a T cell receptor, or a B-cell receptor. In some embodiments, the T cell receptor is 2D2, B8, or bdc2.5. In some embodiments, the extracellular antigen binding domain comprises a single chain variable fragment (scFV). In some embodiments, the extracellular antigen binding domain comprises an scFv of 2D2 or B8. In some embodiments, the intracellular domain comprises one or more costimulatory domains. In some embodiments, the one or more costimulatory domains are selected from a CD28 costimulatory domain, a CD3ζ-chain, a 4-1BBL costimulatory domain, or any combination thereof. In some embodiments, the nucleic acid encoding the TCR is operably linked to an inducible promoter or a conditional promoter.

Also described herein are methods for preparing an engineered CD8+ T cell, comprising transducing cytotoxic CD8+ T cells with a nucleic acid encoding a T cell receptor that binds to a self-antigen bound to a major histocompatibility complex (MHC) class II.

Also described herein are methods for preparing an engineered CD8+ T cell, comprising transducing cytotoxic CD8+ T cells with a nucleic acid encoding a T cell receptor from isolated CD4+ T cells or a chimeric antigen receptor comprising an antigen binding domain thereof, wherein CD4+ T cells the bind to a self-antigen from a subject having an autoimmune disease. Also, described herein are methods for preparing an engineered T cell comprising: (a) isolating CD4+ T cells that bind to a self-antigen from a subject having an autoimmune disease; (b) isolating nucleic acid encoding a T cell receptor from the isolated CD4+ T cells; and (c) transducing cytotoxic CD8+ cells with nucleic acid encoding the T cell receptor from the isolated CD4+ T cells or a chimeric antigen receptor comprising an antigen binding domain thereof. In some embodiments, the CD4+ T cells are MHC Class II-restricted T cells. In some embodiments, the methods further comprise expanding the transduced CD8+ cells. In some embodiments, expanding the transduced CD8+ cells comprise stimulation with an anti-CD3 and/or an anti-CD28 antibody.

In any accordance with any of the foregoing embodiments, the methods may further comprise administering the transduced CD8+ cells to a subject in need thereof. In some embodiments, the subject has an autoimmune disease or condition. In some embodiments, the autoimmune disease is multiple sclerosis or Type I diabetes. In some embodiments, the subject is a human subject.

Also described herein are methods for treating an autoimmune disease or condition, comprising administering an engineered CD8+ T cell as described herein to a subject in need thereof. In some embodiments, the autoimmune disease or condition is multiple sclerosis. In some embodiments, the autoimmune disease or condition is Type I diabetes. In some embodiments, the subject is a human subject.

In accordance with any of the foregoing treatment embodiments, administering the engineered CD8+ T cell may decrease activation of CD4+ cells in the subject, decreases tissue damage in the subject, and/or decreases autoimmune inflammation in the subject compared to no administration of the engineered CD8+ T cell.

In accordance with any of the foregoing treatment embodiments, the methods may further comprise administering one or more additional therapeutic agents. In some embodiments, the one or more additional therapeutic agents is an anti-inflammatory agent or an immunosuppressive agent.

In accordance with any embodiments described herein, the CD8+ T cells may be derived from an autologous donor or an allogenic donor.

Also described herein are engineered CD8+ T cells for use in treating an autoimmune disease as described herein, and uses of engineered CD8+ T cells in the preparation of medicaments for treating an autoimmune disease as described herein.

### BRIEF DESCRIPTION OF THE DRAWINGS

FIG. 1 illustrates that MHC Class II TCR can be expressed in mature CD8+ T cells. Engineered CD8+ T cells were produced using CD8+ spleen and lymph node cells using retrovirus encoding an OT-II or 2D2 TCR. Cells were examined for transduction efficiency by expression of the transgenic TCR (A) or by expression of GFP (B).

FIG. 2 illustrates an exemplary protocol for assaying specific lysis of peptide pulsed splenocytes by engineered CD8+ T cells.

FIG. 3 illustrates that engineered CD8+ T cells do not produce cytokines in response to antigen stimulation through the transgenic TCR. B8 engineered CD8+ T cells were incubated with or without a 50 μM concentration of the peptide recognized by the B8 TCR for 16 hours and then examined for production of interferon gamma. No significant expression of cytokine was shown following stimulation.

FIG. 4 illustrates modulation of active experimental autoimmune encephalomyelitis (EAE) by engineered CD8+ T cells. MOG₃₅₋₅₅/CFA injected mice were administered 5×10⁶ CD8+ T cells engineered to express B8 or OT-II TCRs or only given diluent at two days after onset of EAE symptoms (Day 0). Clinical scores were followed for 18 days (1: Limp tail; 2: Hind limb weakness/delayed righting reflex; 3: Single hind-limb paralysis; 4: Both hind-limbs paralyzed; 5: Euthanization (front limb paralysis, 20% weight loss, inability to feed). Data shown is mean+/−SEM, *=P<0.05 compared to control by ANOVA of Area Under the Curve (AUC); n≥8 mice/group.

FIG. 5 illustrates continued clinical benefit and CNS localization of B8-expressing CD8+ T cells at 17 days post transfer. MOG35-55/CFA injected mice were administered 5×10⁶ CD8+ T cells engineered to express B8 TCR at two days after onset of EAE symptoms. Clinical scores were followed for 17 days revealing a bimodal response with some mice demonstrating a relapse of disease and others a continued reduction in disease (A). The CNS from individual mice was examined for the presence of adoptively transferred cells *CD45.1+ cells) (B). The engineered B8 CD8+ T cells were increased greater than 10-fold in non-relapsing mice when compared to mice that relapsed. Data shown is representative of 2 non-relapsing and 5 relapsing mice.

FIG. 6 illustrates modulation of active experimental autoimmune encephalomyelitis (EAE) by B8 engineered CD8+ T cells using serial administration. Injections of 5×10⁶ fresh B8 cells were performed on day 0 and twice more on Day? and 14. *p≤0.05 by individual t-test time point comparison, p≤0.005 for combined data by Wilcoxon matched-pairs signed rank test, N=9 for B8 and 5 for Diluent control.

### DETAILED DESCRIPTION

Described herein are CD8+ T cells engineered to express a heterologous T cell receptor specific for an auto-antigen (self-antigen) bound to MHC class II, and methods for using the same, such as in adoptive cell therapy. The engineered CD8+ T cells, which also are referred to as Chimeric Hunters for Antigen Surveillance and Elimination (CHASE), recognize and eliminate APCs displaying self-antigen bound to MHC class II. The engineered CD8+ T cells thus inhibit CD4 T cell effector function by preventing activation of pathogenic self-antigen-specific T cells. Accordingly, the engineered CD8+ T cells are useful for the treatment of autoimmune diseases, including neuroinflammatory diseases (e.g., multiple sclerosis), and Type I diabetes. The engineered CD8+ T cells described herein are highly specific for self-antigen-displaying (APCs) and so are not expected to induce general immunosuppression. One autoimmune disease that can be treated with the engineered CD8+ T cells described herein is the neuroinflammatory disease Multiple Sclerosis (MS), which is a chronic demyelinating disease of the central nervous system (CNS). MS patients often have a relapsing-remitting or progressive disease course. Therapeutic treatments targeting immune cell activity in relapsing-remitting MS as described herein can ameliorate disease and can also benefit primary progressive disease. Other neuroinflammatory diseases also can be treated with the engineered CD8+ T cells described herein, as can other autoimmune diseases, such as Type I diabetes.

### I. Definitions

Technical and scientific terms used herein have the meanings commonly understood by one of ordinary skill in the art to which the present disclosure pertains, unless otherwise defined.

As used herein, the singular forms “a,” “an,” and “the” designate both the singular and the plural, unless expressly stated to designate the singular only.

As will be understood by one skilled in the art, all ranges disclosed herein also encompass any and all possible subranges and combinations of subranges thereof. As will also be understood by one skilled in the art all language such as “up to,” “at least,” “greater than,” “less than,” and the like, include the number recited and refer to ranges which can be subsequently broken down into subranges as discussed above. Further, as will be understood by one skilled in the art, a range includes each individual member. Thus, the range 1-3 as pertaining to discrete items refers to each of 1, 2, and 3 items, while the range 1-3 as pertaining to continuous values refers to all values from 1 to 3 inclusive.

As used herein, the term “about” means that the modified value can vary+/−10% from the stated value without departing from the disclosure.

As used herein, the terms “patient,” “subject,” “individual,” and the like are used interchangeably and denotes any mammal, including humans. For example, a subject may be suffering from or suspected of having an autoimmune disease. In some embodiments the patient, subject or individual is an animal, such as, but not limited to, domesticated animals, such as equine, bovine, murine, ovine, canine, and feline animals.

The terms “administer,” “administration,” or “administering” as used herein refer to (1) providing, giving, dosing and/or prescribing, such as by either a health professional or his or her authorized agent or under his direction, and (2) putting into, taking or consuming, such as by a health professional or the subject. Administration can be carried out by any suitable route, including intravenously, intramuscularly, intraperitoneally, or subcutaneously. Administration includes self-administration and the administration by another.

The terms “treat”, “treating” or “treatment”, as used herein, include alleviating, abating or ameliorating a disease or condition or one or more symptoms thereof, whether or not the disease or condition is considered to be “cured” or “healed” and whether or not all symptoms are resolved. The terms also include reducing or preventing progression of a disease or condition or one or more symptoms thereof, impeding or preventing an underlying mechanism of a disease or condition or one or more symptoms thereof, and achieving any therapeutic benefit. With respect to an autoimmune disease, “treating” or “treatment” also encompasses reducing or inhibiting an immune response, reducing or inhibiting inflammation, reducing or inhibiting one or more symptoms of inflammation, inhibiting or decreasing the risk of relapse of the autoimmune disease and/or maintaining remission and/or absence of symptoms of the autoimmune disease.

“Autoantigen” or “self-antigen” as used herein refers to an immunogenic antigen or epitope which is native to the subject and which may be involved in the pathogenesis of an autoimmune disease.

As used herein, the terms “CD,” “cluster of differentiation” or “common determinant” refer to cell surface molecules recognized by antibodies. Expression of some CDs is specific for cells of a particular lineage or maturational pathway, and the expression of others varies according to the state of activation, position, or differentiation of the same cells.

As used herein, “immune-related disease” means a disease in which the immune system is involved in the pathogenesis of the disease. A subset of immune-related diseases is autoimmune diseases. Autoimmune diseases include, but are not limited to, multiple sclerosis, rheumatoid arthritis, myasthenia gravis, psoriasis, systemic lupus erythematosus, autoimmune thyroiditis (Hashimoto's thyroiditis), Graves' disease, inflammatory bowel disease, autoimmune uveoretinitis, polymyositis, and certain types of diabetes, including Type 1 diabetes.

As used herein, the term “immune cell” refers to any cell that plays a role in the immune response. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes.

The term “lymphocyte” refers to all immature, mature, undifferentiated and differentiated white lymphocyte populations including tissue specific and specialized varieties. It encompasses, by way of non-limiting example, B cells, T cells, NKT cells, and NK cells. As used herein, the term T-cell includes naive T cells, CD4+ T cells, CD8+ T cells, memory T cells, activated T cells, anergic T cells, tolerant T cells, chimeric T cells, and antigen-specific T cells.

As used herein “adoptive cell therapeutic composition” refers to any composition comprising cells suitable for adoptive cell transfer. In exemplary embodiments, the adoptive cell therapeutic composition comprises a cell type selected from TCR (i.e. heterologous T-cell receptor), modified lymphocytes, and CAR (i.e. chimeric antigen receptor) modified lymphocytes. In another embodiment, the adoptive cell therapeutic composition comprises a cell type selected from T-cells, CD8+ cells, CD4+ cells, NK-cells, delta-gamma T-cells, regulatory T-cells and peripheral blood mononuclear cells. In another embodiment, one or more of T-cells, CD8+ cells, CD4+ cells, NK-cells, delta-gamma T-cells, regulatory T-cells or peripheral blood mononuclear cells are comprised in an adoptive cell therapeutic composition. In one embodiment, the adoptive cell therapeutic composition comprises CD8+ cells engineered to express a heterologous T-cell receptor as described herein.

As used herein, “engineered CD8+ cells” refers to CD8+ cells that contain heterologous nucleic acid encoding a T cell receptor (TCR) that binds to a self-antigen in a complex with MHC class II. In some embodiments, the TCR is a native TCR. In some embodiments, the TCR is a modified native TCR having one or more amino acid substitutions or deletions as compared to a native TCR. In some embodiments, the TCR is a chimeric antigen receptor as described herein.

### II. Overview

The present disclosure relates in part to the treatment of autoimmune diseases by administering a composition comprising one or more CD8+ T cells engineered to express an autoantigen-MHC class II-specific receptor. The methods provided herein are based in part on the use of targeted destruction of MHC class II positive cells to block CD4 activation without damaging target tissue. CD4+ cells are generally necessary for autoimmunity and require autoantigen-MHC class II recognition on site for activation. Accordingly, CD4+ cells can express TCRs that recognize autoantigen bound by MHC class II. The MHC class II can be found on dendritic cells, macrophages, monocytes, microglial cells, and astrocytes, but typically not on other cells, such as neurons or oligodendrocytes in MS lesions or beta islet cells in diabetes. As described herein, these autoreactive CD4+ T cells can be isolated, and the nucleic acids encoding the MHC II-restricted autoantigen receptors can be cloned. The nucleic acids encoding the TCRs can then be introduced into CD8+ T cells to generate a cytotoxic T cell that specifically targets APCs expressing the autoantigen in the context of MHC class II. Given the high specificity for autoantigen expressed by an APC, the methods are expected to cause minimal general immunosuppression. The engineered CD8+ T cells are useful for the treatment of a variety of autoimmune diseases, including, but not limited to, multiple sclerosis (MS) and diabetes (including Type 1 diabetes).

TCRs are heterodimers composed of two chains which can be αβ (alpha-beta) or γδ (gamma-delta). The structure of TCRs is very similar to that of immunoglobulins (Ig). Each chain has two extracellular domains, which are immunoglobulin folds. The amino-terminal domain is highly variable and called the variable (V) domain. The domain closest to the membrane is the constant (C) domain. These two domains are analogous to those of immunoglobulins, and resemble Fab fragments. The V domain of each chain has three complementarity determining regions (CDR). Proximal to the membrane, each TCR chain has a short connecting sequence with a cysteine residue that forms a disulfide bond between both chains.

Genes encoding αβ and γδ heterodimers are only expressed in the T-cell lineage. The four TCR loci (α, β, γ and δ) have a germ-line organization very similar to that of Ig. α and γ chains are produced by rearrangements of V and J segments whereas β and δ chains are produced by rearrangements of V, D, and J segments. The gene segments for TCR chains are located on different chromosomes, except the δ-chain gene segments that are between the V and J gene segments of the a chain. The location of δ-chain gene segments has a significance: a productive rearrangement of α-chain gene segments deletes C genes of the δ-chain, so that in a given cell the αβ heterodimer cannot be co-expressed with the γδ receptor.

In mice, there are about 100 Va and 50 Ja genes segments and only one Ca segment. The δ-chain gene family has about 10 V, 2 D, and 2 J gene segments. The β-chain gene family has 20-30 V segments and two identical repeats containing 1 DP, 6 Jβ and 1 CP. Finally, the γ-chain gene family contains 7 V and 3 different J-C repeats. In humans the organization is similar to that of mice, but the number of segments varies.

The rearrangements of gene segments in a and (3 chains is similar to that of Igs. The a chain, like the light chain of Ig is encoded by V, J, and C gene segments. The (3 chain, like the heavy chain of Ig, is encoded by V, D, and J gene segments. Rearrangements of a chain gene segments result in VJ joining and rearrangements of (3 chain result in VDJ joining. After transcription of rearranged genes, RNA processing, and translation, the a and (3 chains are expressed linked by a disulfide bond in the membrane of T cells.

TCR gene segments are flanked by recognition signal sequences (RSS) containing a heptamer and a nonamer with an intervening sequence of either 12 nucleotides (one turn) or 23 nucleotides (two turn). As in Igs, enzymes encoded by recombination-activating genes (RAG-1 and RAG-2) are responsible for the recombination processes. RAG1/2 recognize the RSS and join V-J and V-D-J segments in the same manner as in Ig rearrangements. Briefly, these enzymes cut one DNA strand between the gene segment and the RSS and catalyze the formation of a hairpin in the coding sequence. The signal sequence is subsequently excised.

The combinatorial joining of V and J segments in a chains and V, D and J segments in β chains produces a large number of possible molecules, thereby creating a diversity of TCRs. Diversity is also achieved in TCRs by alternative joining of gene segments. In contrast to Ig, β and δ gene segments can be joined in alternative ways. RSS flanking gene segments in β and δ gene segments can generate VJ and VDJ in the β chain, and VJ, VDJ, and VDDJ on the δ chain. As in the case of Ig, diversity is also produced by variability in the joining of gene segments.

Hypervariable loops of the TCR known as complementarity determining regions (CDRs) recognize the composite surface made from a MHC molecule and a bound peptide. The CDR2 loops of α and β contact only the MHC molecule on the surface of APC, while the CDR1 and CDR3 loops contact both the peptide and MHC molecule. Compared with Ig, TCRs have more limited diversity in the CDR1 and CDR2. However, diversity of the CDR3 loops in TCRs is higher than that of Ig, because TCRs can join more than one D segment leading to augmented junctional diversity.

### III. Isolation of Self-Antigen-MHC II Reactive T Cells

The methods described herein involve the isolation CD4+ T cells that are autoreactive with self-antigens.

Methods for the isolation of autoreactive T cell populations are known in the art and are described in, for example, U.S. Pat. Nos. 7,658,926 and 7,972,848 and US Patent Application Publication 2010/0239548, which are incorporated herein by reference in their entirety. For example, in some embodiments, mononuclear cells are isolated from the cerebrospinal fluid (CSFMCs) or peripheral blood (PBMCs) of a patient having an autoimmune disease. Mononuclear cells can be enriched in the sample by using centrifugation techniques known to those in the art, including, but not limited to, Ficoll® gradients. Further CD4+ cells can be enriched using CD4+ cell surface markers by cell sorting techniques, such as fluorescence-activated cell sorting (FACS).

The isolated mononuclear cells can then be cultured with a peptide antigen that comprises an epitope present in the self-antigen. In some embodiments, the APCs in the isolated sample are sufficient for presentation of the antigen in the context of an MHC class II. In some embodiments exogenous APCs are added to the culture.

Generally, isolated mononuclear cells are incubated with the self-antigen for a time sufficient to activate self-antigen-reactive T cells. In some embodiments, the cells are incubated with the self-antigen for 7-10 days. Cultures are then tested for specific proliferation to self-antigens, such as by measuring [³H]-thymidine incorporation in the presence of the self-antigen over a period of about 1-7 days, such as about 1, 2, 3, 4, 5, 6, or 7 days. Cultures testing positive for specific proliferation to self-antigen can be serially diluted to obtain clonal T cell lines or maintain as oligoclonal cultures. The cells can be cultured for about 4 to about 8 weeks to expand the T cells. When the T cells are clonal, the T cells are homogenous with a single pattern of Vβ-Dβ-Jβ gene usage for the TCR. The genes encoding the α and β chains of the TCR and/or fragments thereof can then be isolated using known recombinant techniques. In some embodiments, the cultures are oligoclonal and genes encoding the α and β chains of the TCR and/or fragments thereof are cloned from the oligoclonal cells.

In some embodiments, the self-antigen is associated with an autoimmune disease, such as an antigenic peptide associated with an autoimmune disease. Exemplary self-antigens are disclosed, for example, in US Patent Application Publication 2016/0022788, which is incorporated herein by reference in its entirety.

In some embodiments, the self-antigen is an antigenic peptide of or derived from myelin oligodendrocyte glycoprotein (MOG), myelin basic protein (MBP), myelin associated glycoprotein (MAG), alphaB-crystallin, S100beta, or proteolipid protein (PLP). In some embodiments, myelin basic protein (MBP), myelin associated glycoprotein (MAG), alphaB-crystallin, S100beta, proteolipid protein (PLP) and myelin oligodendrocyte glycoprotein (MOG) have the following sequences: Myelin oligodendrocyte glycoprotein (human; GenBank CAA52617.1); Myelin oligodendrocyte glycoprotein (mouse; GenBank: AAH80860.1); Myelin basic protein (human; Accession No: P02686); Myelin basic protein (mouse; GenBank: AAB59711.1); Myelin associated glycoprotein (human; GenBank: AAH53347.1); Myelin associated glycoprotein (mouse; Accession No.: P20917); S100beta (human; Accession No.: NP006263); S100beta (mouse; Accession No.: NP033141); Proteolipid protein (human; GenBank: AAA60117.1); Proteolipid protein (mouse; GenBank: CAA30184.1); AlphaB crystallin (human; Accession No.: 2KLR_A); and AlphaB crystallin (mouse; GenBank: AAH94033.1).

In particular embodiments, the self-antigen is MOG or a fragment, variant, analog, homolog or derivative thereof. In some embodiments, the self-antigen is a fragment of MOG of between 4 and 50 amino acids that comprises residues 35-55 of MOG or a fragment, variant, analog, homolog or derivative thereof. In some embodiments, the fragment of MOG is 4, 6, 8, 10, 12, 15, 20, 25, 30, 35, 40, 45 or 50 amino acids in length. In some embodiments, the self-antigen is a fragment of MOG comprising amino acids 35-55 or a fragment, variant, analog, homolog or derivative thereof. In some embodiments, the self-antigen is a peptide consisting of amino acids 35-55 of MOG or a fragment, variant, analog, homolog or derivative thereof.

In particular embodiments, the self-antigen is MBP or a fragment, variant, analog, homolog or derivative thereof. In some embodiments, the self-antigen is a fragment of MBP of between 4 and 50 amino acids that comprises residues 96-102 of MBP or a fragment, variant, analog, homolog or derivative thereof. In some embodiments, the fragment of MBP is 4, 6, 8, 10, 12, 15, 20, 25, 30, 35, 40, 45 or 50 amino acids in length. In some embodiments, the self-antigen is a fragment of MBP comprising amino acids 93-105 or a fragment, variant, analog, homolog or derivative thereof. In some embodiments, the self-antigen is a peptide consisting of amino acids 93-105 of MBP or a fragment, variant, analog, homolog or derivative thereof.

In some embodiments, the autoimmune disease associated self-antigen is selected from MOG35-55 mouse fragment, MEVGWYRSPFSRVVHLYRNGK (SEQ ID NO: 1); MOG human fragment, MEVGWYRPPFSRVVHLYRNGK (SEQ NO:2); MAG287-295 human fragment, SLLLELEEV (SEQ NO:3); MAG287-295 mouse fragment, SLYLDLEEV (SEQ NO:4); MAG509-517 mouse and human fragment, LMWAKIGPV (SEQ NO:5); MAG556-564, human fragment, VLFSSDFRI (SEQ NO:6); MAG556-564, mouse fragment, VLYSPEFRI (SEQ NO:7); MBP human fragment, SLSRFSWGA (SEQ NO:8); MBP mouse fragment, SLSRFSWGG (SEQ NO:9); MOG mouse and human fragment, KVEDPFYWV (SEQ NO:10); MOG mouse and human fragment, RTFDPHFLR (SEQ NO:11); MOG mouse and human fragment, FLRVPCWKI (SEQ NO:12); MOG mouse and human fragment, KITLFVIVPV (SEQ NO:13); MOG mouse and human fragment, VLGPLVALI (SEQ NO:14); MOG mouse and human fragment, TLFVIVPVL (SEQ NO:15); MOG mouse and human fragment, RLAGQFLEEL (SEQ NO:16); PLP80-88 mouse and human fragment, FLYGALLLA (SEQ NO:17); MOG fragment, HPIRALVGDEVELP (SEQ NO:18); MOG fragment, VGWYRPPFSRVVHLYRNGKD (SEQ NO:19); MOG fragment LKVEDPFYWVSPGVLVLLAVLPVLLL (SEQ NO:20); and combinations thereof.

In some embodiments, the autoimmune disease associated self-antigen is a diabetes mellitus-associated antigen. In some embodiments, the self-antigen is selected from insulin (GenBank: AAA59172.1), chromogranin A (GenBank: AAB53685.1), glutamic acid decarboxylase (GAD1; GAD67 GenBank: CAB62572.1), glutamate decarboxylase 2 (NCBI: NP_032104.2; GAD2; GAD65) and islet-specific glucose-6-phosphatase catalytic subunit-related protein (GenBank: AAF82810.1) and combinations thereof. Antigenic fragments and antigenic derivatives of these antigens are also contemplated. In some embodiments, the antigen can be proinsulin. In some embodiments, the proinsulin antigen can have the sequence MALWMRLLPLLALLALWGPDPAAAFVNQHLCGSHLVEALYLVCGERGFF YTPKTRREAEDLQVGQVELGGGPGAGSLQPLALEGSLQKRGIVEQCCTSICS LYQLENYCN (SEQ NO:21), which can be encoded by a sequence contained in GenBank Accession No. NM000207, the contents of which are incorporated by reference herein. In some embodiments, the insulin antigen comprises the sequence MRLLPLLALLA (SEQ NO:22), SHLVEALYLVCGERG (SEQ NO:23), or LYLVCGERG (SEQ NO:24). In some embodiments, the insulin antigen can have the amino acid sequence GIVEQCCTSICSLYQ (SEQ NO:25). Combinations of the above listed antigens are also contemplated.

In some embodiments, the autoimmune disease associated self-antigen is a rheumatoid arthritis associated antigen. In some embodiments, the rheumatoid arthritis associated self-antigen can be the peptide (Q/R)(K/R)RAA (SEQ NO:26). In some embodiments, the arthritis associated self-antigen can be type II collagen or a fragment thereof. In some embodiments, the type II collagen fragment is selected from the group consisting of AGERGPPG (SEQ NO:27), AGGFDEKAGGAQLGV (SEQ NO:28), VGPAGGPGFPG (SEQ NO:29), and a combination thereof.

In some embodiments, the autoimmune disease associated self-antigen is a myocarditis associated self-antigen. In some embodiments, the myocarditis associated self-antigen is myosin or an antigenic fragment or antigenic derivative. In some embodiments, the antigen can be a peptide contained in human myosin (GeneBank Accession No. CAA86293.1). In some embodiments, the antigen can be a peptide contained within α-myosin, and can have the sequence Ac-SLKLMATLFSTYASADTGDSGKGKGGKKKG (Ac=acetyl group) (SEQ NO:30), GQFIDSGKAGAEKL (SEQ NO:31), DECSELKKDIDDLE (SEQ NO:32), and combinations thereof.

In some embodiments, the autoimmune disease associated self-antigen is a thyroiditis associated antigen. In some embodiments, the self-antigen is selected from thyroid peroxidase (TPO), thyroglobulin, or Pendrin. In some embodiments, the thyroglobulin self-antigen can have the sequence, NIFEXQVDAQPL (SEQ NO:33), YSLEHSTDDXASFSRALENATR (SEQ NO:34), RALENATRDXFIICPIIDMA (SEQ NO:35), LLSLQEPGSKTXSK (SEQ NO:36), EHSTDDXASFSRALEN (SEQ NO:37) and combinations thereof, wherein Xis 3,5,3′,5′-tetraiodothyronine (thyroxine). In some embodiments, the TPO self-antigen can have the sequence LKKRGILSPAQLLS (SEQ NO:38), SGVIARAAEIMETSIQ (SEQ NO:39), PPVREVTRHVIQVS (SEQ NO:40), PRQQMNGLTSFLDAS (SEQ NO:41), LTALHTLWLREHNRL (SEQ NO:42), HNRLAAALKALNAHW (SEQ NO:43), ARKVVGALHQIITL (SEQ NO:44), LPGLWLHQAFFSPWTL (SEQ NO:45), MNEELTERLFVLSNSST (SEQ NO:46), LDLASINLQRG (SEQ NO:47), RSVADKILDLYKHPDN (SEQ NO:48), IDVWLGGLAENFLP (SEQ NO:49) and combinations thereof. The Pendrin self-antigen can have the sequence QQQHERRKQERK (SEQ NO:50) (amino acids 34-44 in human pendrin (GenBank AF030880)), PTKEIEIQVDWNSE (SEQ NO:51) (amino acids 630-643 in human pendrin), or NCBI GenBank Accession No. NP-000432.1.

Cross-reactive T cells can be present in any sample comprising mononuclear cells. For example, the sample can be isolated from the peripheral blood or cerebral spinal fluid of an MS patient, from peripheral blood of a diabetic patient or from the synovial fluid of a RA patient. T cells from patients with other autoimmune diseases can be similarly isolated from peripheral blood and/or tissues involved with the disease.

In some embodiments, the TCR is specific for a self-antigen in a complex with a mammalian MHC class II. In some embodiments, the MHC class II comprises a human MHC class II. In some embodiments the human MHC class II is HLA-DP, HLA-DM, HLA-DOA, HLA-DOB, HLA-DQ, or HLA-DR. In some embodiments, the MHC class II comprises a human MHC class II. In some embodiments the human MHC class II is HLA-DR2, HLA-DR3, HLA-DR4, HLA-DR11, HLA-DR15 or HLA-DQ6. In some embodiments, the MHC class II comprises a murine MHC class II (e.g., H-2A), HLA-DP, HLA-DM, HLA-DOA, HLA-DOB, HLA-DQ, or HLA-DR

### IV. Viral Vectors and Transduction

In some embodiments, nucleic acids encoding the a and chains of the self-antigen-MHC class II-specific TCRs are cloned into suitable vectors for expression in CD8+ cells. In some embodiments, the TCRs can be derived from available TCRs, such as the murine 2D2 low affinity TCR for I-Ab/MOG35-55 and B8: low affinity TCR for I-Ab/MOG35-55, which recognize the MS associated MOG antigen or the BDC2.5 TCR, which recognizes peptides from chromogranin A. In some embodiments, the TCRs can be derived from isolated self-reactive CD4+ T cells as described above.

Many expression vectors are available and known to those of skill in the art and can be used for expression of TCR polypeptides provided herein. The choice of expression vector will be influenced by the choice of host expression system, e.g. CD8+ T cell. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g. a hexa-his tag or a myc tag, hemagglutinin tag or a tag for purification, for example, a GST fusion, and a sequence for directing protein secretion and/or membrane association.

Any methods known to those of skill in the art for the insertion of DNA fragments into a vector can be used to construct expression vectors containing a nucleic acid encoding any of the TCR polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Genetic modification of engineered CD8+ T cells can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the TCR can be cloned into a retroviral vector and expression can be driven from its endogenous promoter, from the retroviral long terminal repeat, or from an alternative internal promoter.

For initial genetic modification of the CD8+ cells to provide self-antigen-MHC II targeted TCRs, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al. Mol. Cell. Biol. 5:431-437 (1985)); PA317 (Miller, et al. Mol. Cell. Biol. 6:2895-2902 (1986)); and CRIP (Danos, et al. Proc. Natl. Acad. Sci. USA 85:6460-6464 (1988)). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. Blood 80: 1418-1422 (1992), or culturing with viral supernatant alone or concentrated vector stocks with or without appropriate growth factors and polycations, e.g., by the method of Xu, et al. Exp. Hemat. 22:223-230 (1994); and Hughes, et al. J. Clin. Invest. 89: 1817 (1992).

Transducing viral vectors can be used to express a co-stimulatory ligand and/or secrete a cytokine (e.g., 4-1BBL and/or IL-12) in an engineered immune cell. Preferably, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natl. Acad. Sci. U.S.A. 94: 10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244: 1275-1281 (1989); Eglitis et al., BioTechniques 6:608-614, (1988); Tolstoshev et al., Current Opinion in Biotechnology 1:55-61 (1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing a presently disclosed TCRs is a retroviral vector, e.g., an oncoretroviral vector.

Non-viral approaches can also be employed for the expression of a protein in cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J. Med. Sci. 298:278, (1989); Staubinger et al., Methods in Enzymology 101:512 (1983)), asialoorosomucoid-polylysine conjugation (Wu et al., Journal of Biological Chemistry 263: 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

CD8+ T cells for transduction can be obtain from any available source. In some embodiments the CD8+ cells are obtained from a donor subject, transduced with the self-antigen-MI-IC II specific TCR and inject back into same subject (i.e. autologous transfer). In some embodiments the CD8+ cells are obtained from a donor subject, transduced with the self-antigen-MHC II specific TCR and inject into different subject (i.e. allogenic transfer).

In some embodiments, the transduced CD8+ T cells are expanded prior to administration to a subject. In some embodiments, the transduced CD8+ T cells are expanded in the presence of αCD3 and/or αCD28.

### V. Chimeric Antigen Receptors (CAR) and CAR T Cells

In some embodiments, the nucleic acids encoding the antigen binding portions of the self-reactive TCRs are used to generate chimeric antigen receptors. In some embodiments, the engineered CD8+ cells provided herein express at least one chimeric antigen receptor (CAR). There are currently three generations of CARs.

In some embodiments, the engineered CD8+ cells provided herein express a “first generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD3ζ chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation.

In some embodiments, the engineered CD8+ cells provided herein express a “second generation” CAR. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-IBB) and activation (e.g., CD3ζ). Preclinical studies have indicated that “Second Generation” CARs can improve the antitumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL).

In some embodiments, the engineered CD8+ cells provided herein express a “third generation” CAR. “Third generation” CARs comprise those that provide multiple co-stimulation (e.g., CD28 and 4-1BB) and activation (e.g., CD3). 10831 In accordance with the presently disclosed subject matter, the CARs of the engineered CD8+ cells provided herein comprise an extracellular antigen-binding domain, a transmembrane domain and an intracellular domain.

Nucleic acids encoding the CARs can be inserted in a vector for transduction and expression in CD8+ T cells as described above.

### VI. Therapeutic Methods

Provided herein are methods for the treatment of an autoimmune disease involving administration of a composition contain one or more engineered CD8+ cells described herein, wherein the engineered CD8+ T cells recognize a self-antigen bound to an MEW class II, wherein the self-antigen is associated with the autoimmune disease. The engineered CD8+ T cells described herein can be used in different therapeutic methods, including methods of treating or ameliorating autoimmune disease in a subject in need thereof, methods of increasing or lengthening survival of a subject having an autoimmune disease, and methods for treating or preventing or reducing the risks of an autoimmune disease or one or more symptoms thereof in a subject in need thereof. The methods may comprise administering an effective amount of the engineered CD8+ T cells to the subject. In some embodiments, the subject is resistant to one or more conventional therapies for the treatment of an autoimmune disease.

Target autoimmune diseases include, but are not limited to, neuroinflammatory diseases, such as multiple sclerosis, and other autoimmune diseases, such as diabetes (including Type 1 diabetes, diabetes mellitus), experimental autoimmune encephalomyelitis (EAE), transplantation rejection, premature ovarian failure, scleroderma, Sjogren's disease, lupus, vitiligo, alopecia (baldness), polyglandular failure, Grave's disease, hypothyroidism, polymyositis, pemphigus, autoimmune hepatitis, hypopituitarism, myocarditis, thyroiditis, Addison's disease, autoimmune skin diseases, uveitis, pernicious anemia, hypoparathyroidism, and/or rheumatoid arthritis.

Self-antigens associated with autoimmune diseases are known, and include those set forth above and below. For example, for MS, self-antigens include myelin oligodendrocyte glycoprotein (MOG), myelin basic protein (MBP), myelin associated glycoprotein (MAG), proteolipid protein (PLP), and fragments thereof, such as MOG₃₅₋₅₅. For diabetes, self-antigens include glutamic acid decarboxylase (GAD67), glutamic acid decarboxylase 2 (GAD65), insulin, chromogranin A, islet-specific glucose-6-phosphatase catalytic subunit-related protein, and fragments thereof.

As noted above, an effective amount of the engineered CD8+ T cells is an amount determined to be effective in producing the desired effect, for example, treatment of an autoimmune disease or condition and/or amelioration of one or more symptoms of an autoimmune disease or condition, even if the amount is not effective in the specific patient treated. An effective amount can be provided in one administration (one dose) or in a series of administrations. An effective amount can be provided in a bolus administration or by continuous perfusion. In some embodiments, for adoptive immunotherapy using antigen-specific T cells, cell doses in the range of about 10⁶ to about 10¹⁰ are infused.

In some embodiments, an effective amount of engineered CD8+ T cells that recognize a multiple sclerosis associated self-antigen bound to an MHC class II is administered for the treatment of multiple sclerosis and/or amelioration of one or more symptoms of multiple sclerosis. In some embodiments the self-antigen is a central nervous system (CNS) antigen. In some embodiments the self-antigen is selected from myelin oligodendrocyte glycoprotein (MOG), myelin basic protein (MBP), myelin associated glycoprotein (MAG), proteolipid protein (PLP) and fragments thereof. In some embodiments the self-antigen is MOG₃₅₋₅₅.

In some embodiments, an effective amount of engineered CD8+ T cells that recognize a diabetes associated self-antigen bound to an MHC class II is administered for the treatment of diabetes and/or amelioration of one or more symptoms of diabetes. In some embodiments, the diabetes associated antigen is selected from glutamic acid decarboxylase (GAD67), glutamic acid decarboxylase 2 (GAD65), insulin, chromogranin A, islet-specific glucose-6-phosphatase catalytic subunit-related protein and fragments thereof.

In some embodiments, an effective amount of engineered CD8+ T cells that recognize a rheumatoid arthritis associated self-antigen bound to an MHC class II is administered for the treatment of rheumatoid arthritis and/or amelioration of one or more symptoms of rheumatoid arthritis. In some embodiments, the rheumatoid arthritis associated antigen is type II collagen or a fragment thereof.

In some embodiments, an effective amount of engineered CD8+ T cells that recognize a myocarditis associated self-antigen bound to an MHC class II is administered for the treatment of myocarditis and/or amelioration of one or more symptoms of myocarditis. In some embodiments, the myocarditis associated antigen is myosin or a fragment thereof.

In some embodiments, an effective amount of engineered CD8+ T cells that recognize a thyroiditis associated self-antigen bound to an MHC class II is administered for the treatment of thyroiditis and/or amelioration of one or more symptoms of thyroiditis. In some embodiments, the thyroiditis associated antigen is selected from thyroid peroxidase (TPO), thyroglobulin, Pendrin, and fragments thereof.

Engineered CD8+ T cells expressing a self-antigen-MHC II specific TCR can be provided systemically or directly to a subject for treating or preventing or reducing the risks of an autoimmune disease. In certain embodiments, engineered CD8+ T cells are directly injected into tissue of interest (e.g., an organ affected by autoimmune inflammation) or indirectly by administration into the circulatory system. Expansion and differentiation agents can be provided prior to, during or after administration of cells and compositions to increase production of T cells in vitro or in vivo.

Engineered CD8+ T cells as described herein can be administered in any physiologically acceptable vehicle, systemically or regionally, normally intravascularly, intraperitoneally, intrathecally, or intrapleurally, although they may also be introduced into a site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising engineered CD8+ T cells can comprise a purified population of cells. The percentage of engineered CD8+ T cells in a cell population can be determined using various known methods, such as fluorescence activated cell sorting (FACS). The ranges of purity in cell populations comprising engineered CD8+ T cells can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. The engineered CD8+ T cells can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions as described herein comprise pharmaceutical compositions comprising engineered CD8+ T cells expressing self-antigen-MHC II specific TCR with a pharmaceutically acceptable carrier. Administration can be autologous or non-autologous. For example, CD8+ T cells can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising engineered CD8+ T cells expressing self-antigen-MHC II specific TCR), it can be formulated in a unit dosage injectable form (solution, suspension, emulsion).

One consideration concerning the therapeutic use of the engineered CD8+ T cells of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ engineered CD8+ T cells of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more engineered CD8+ T cells of the presently disclosed subject matter are administered to a human subject. The precise determination of what would be considered an effective dose may be based on factors individual to each subject, including their size, age, sex, weight, and condition of the particular subject.

For any composition to be administered to an animal or human, and for any particular method of administration, toxicity may be determined, such as by determining the lethal dose (LD) and LD50 in a suitable animal model e.g., rodent such as mouse; and, the dosage of the composition(s), concentration of components therein and timing of administering the composition(s), which elicit a suitable response.

As noted above, the engineered CD8+ T cells described herein can be administered by any suitable means, including, but not limited to, pleural administration, intravenous administration, subcutaneous administration, intranodal administration, intrathecal administration, intrapleural administration, intraperitoneal administration, and direct administration to the thymus. In certain embodiments, the engineered CD8+ T cells and the compositions comprising thereof are intravenously administered to the subject in need. The engineered CD8+ T cells described herein can be administered by methods used for administering cells for adoptive cell therapies, including, for example, donor lymphocyte infusion and CAR T cell therapies.

In some embodiments, the engineered CD8+ cells are administered in combination with one or more therapeutic agents. In some embodiments, the one or more additional therapeutic agents include an anti-inflammatory agent or an immunosuppressive agent. In some embodiments, the additional therapeutic agent is interferon beta, glatiramer acetate, dimethyl fumarate, teriflunomide, mitoxantrone, fingolimode, daclizumab (anti-CD25), alemtuzumab (anti-CD52), natalizumab (anti-CD49d) or ocrelizumab (anti-CD20). The engineered CD8+ cells can be administered simultaneously or sequentially with the one or more other therapeutic agents, in any order.

Upon administration of the engineered CD8+ T cells into the subject, the engineered CD8+ T cells may be induced that are specifically directed against self-antigen. As used herein, “induction” of T cells includes inactivation of antigen-specific T cells such as by deletion or anergy. Inactivation is particularly useful to establish or reestablish tolerance such as in autoimmune disorders.

In some embodiments, administering the engineered CD8+ T cell decreases antigen-specific activation of CD4+ cells in the subject, decreases tissue damage in the subject, and/or decreases autoimmune inflammation in the subject compared to no administration of the engineered CD8+ T cell.

Also provided herein are engineered CD8+ T cells as described herein for use in treating an autoimmune disease as described herein, and uses of engineered CD8+ T cells as described herein in the preparation of medicaments for treating an autoimmune disease as described herein.

### VII. Articles of Manufacture and Kits

Also provided herein are kits for the treatment or prevention or reduction of risk of an autoimmune disease or one or more symptoms of an autoimmune disease (e.g., autoimmune inflammation). In certain embodiments, the kit comprises a pharmaceutically acceptable composition containing an effective amount of an engineered CD8+ T cell expressing a self-antigen-MHC II specific TCR, optionally in unit dosage form. In particular embodiments, the cells further express at least one co-stimulatory ligand. In some embodiments, the kit comprises a sterile container which contains the composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the engineered CD8+ T cell can be provided together with instructions for administering the engineered cells to a subject in need thereof, as discussed above.

The following examples are provided for illustration purposes only, and are not limiting of the scope of the invention described herein.

### EXAMPLES

### Example 1

These experiments represent a paradigm shifting approach to the treatment of the CD4+ T cell mediated autoimmunity thought to drive pathogenesis in autoimmune disease, using multiple sclerosis as a model autoimmune disease. The methods involve modifying mature cytotoxic T cells to express a transgenic MHC class II/myelin antigen responsive T cell receptor (TCR) and examining the functional consequences of introducing these cells in the context of neuroinflammation.

In this example, the capacity of mature CD8+ T cells modified to recognize myelin antigens in the context of MHC class II molecules to modulate established CD4+ T cell driven autoimmunity was examined. Experimental autoimmune encephalomyelitis (EAE) is the most commonly used experimental model for the human inflammatory demyelinating disease, multiple sclerosis (MS). EAE is a complex condition in which the interaction between a variety of immunopathological and neuropathological mechanisms leads to an approximation of the key pathological features of MS: inflammation, demyelination, axonal loss and gliosis. The counter-regulatory mechanisms of resolution of inflammation and remyelination also occur in EAE, which, therefore can also serve as a model for these processes. Moreover, EAE is often used as a model of cell-mediated organ-specific autoimmune conditions in general. The capacity of engineered CD8+ T cells to ameliorate a variety of EAE models, which are known to require CD4+ T cell activity for disease pathogenesis, was studied herein.

Three TCRs with previously characterized antigen recognition capacities were employed to generate a set of engineered CD8+ T cells for study: 1) commercially available 2D2 TCR, which is a bi-specific murine CD4+ T cell receptor that recognizes myelin oligodendrocyte glycoprotein 35-55 peptide (MOG₃₅₋₅₅) and the neuronal antigen neurofilament medium 15-35 peptide (NF-M15-35) in the context of I-A(b) (murine MHC II) (Bettelli et al. (2003) J Exp. Med. 197: 1073-81; Krishnamoorthy et al. (2009) Nat Med 15: 626-32, 2) B8 TCR, which is a murine CD4+ T cell receptor that also recognizes MOG₃₅₋₅₅; and 3) commercially available OT-II TCR, which is a murine CD4+ T cell receptor that recognizes ovalbumin 323-329 peptide (OVA₃₂₃₋₃₃₉) in the context of I-A(b).

TCRα and TCRβ chain DNA sequences separated by a porcine teschovirus-1 P2A peptide sequence were cloned, using PCR amplification, into a retroviral plasmid vector, pMSCV-IRES-GFP II (pMIG II). The TCRα and TCRβ chains of the 2D2 used in the study contained and αCDR3 sequence of VYFCAVRSYNQG and a βCDR3 sequence of CASSLDPGANT, which differ from the original published sequences of VYFCALRSYNFG and CASSLDCGANP, but were confirmed in Lucca et al. (2014) J Immunol 193: 3267-77.

Ecoptropic retroviral vectors driving expression of the TCR receptors and GFP or only GFP were produced using a viral packaging cell line, concentrated, and examined for infectivity. Briefly, pCGP (1 μg/ul), pEco (1 μg/ul), and pMIGII-TCR containing plasmids were transfected into 293Tcells as a viral packaging line using treatment with Fugene 6 (Promega). Viral stocks were collected and concentrated beginning 24 hours after transfection. Virus was concentrated by centrifugation and examined for infectivity.

CD8+ T cells were isolated using a negative selection strategy with antibodies and magnetic bead-based separation, and then infected with retrovirus while being activated with optimized doses of anti-CD3 and anti-CD28 coated beads. Briefly, murine CD8+ T cells were separated from spleen and lymph nodes by mechanical dissociation of the tissue, followed by treatment with rat monoclonal anti-CD4 and anti-CD24 antibodies, and subsequent treatment with anti-Rat IgG, anti-mouse IgG, and anti-mouse IgM magnetic beads. Negative selection by magnetic separation yielded >80% enriched CD8 T cell populations. The CD8 enriched cells were then activated using anti-CD3 and anti-CD28 stimulation (Dynabeads) In a 24 well tissue culture plate at 1×10⁶ cells/well with pre-washed CD3/CD28 stimulating beads. Cells were incubated in 10% RPMI complete media with rhIL-2 (10 ng/ml) for 24 hrs in a 37° C. 5% CO₂ incubator. Cells were then isolated and transferred to a plate with viral particles pre-bound with 10 ug/ml retronectin and transduction was initiated by centrifugation at RT for 1900 rpm over 5 minutes. Cells and virus were then incubated at 37° C. for 24 hrs with 5% CO₂. After 24 and 72 hours fresh 10% RPMI was added with IL-2 (long/ml). After 6 days resultant transduced cells were isolated by centrifugation with Histopaque 1083 with no brake at 2000 rpm, over 20 min at room temperature and examined for expression of CD8 and GFP.

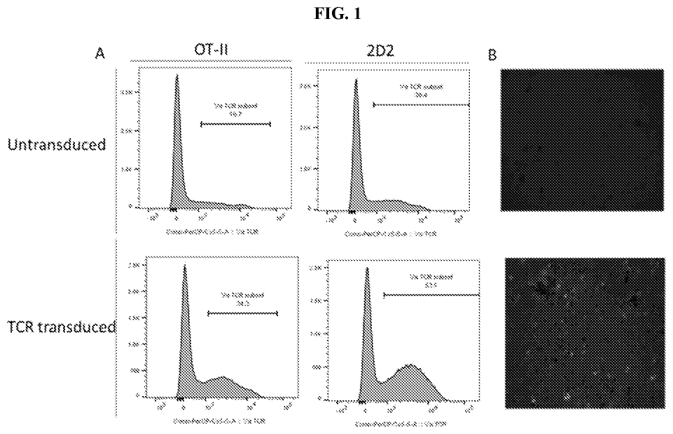

The expression of the transgenic TCRα chains in the 2D2 and OT-II cell populations were examined using flow cytometry (FIG. 1A). TCRα expression in B8 CD8+ T cells was not quantified as no antibody that recognizes the TCR chain was commercially available. However, GFP expression as an indicator of transduction efficiency in all groups was used (representative data shown in FIG. 1B). We found that in all groups CD8 cells could be transduced with an efficiency ranging from 16-25%.

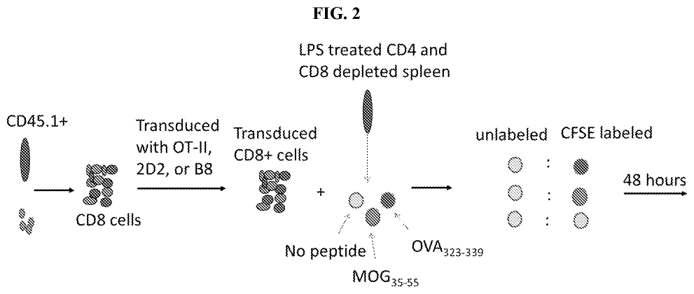

The extent to which expression of the transgenic TCR resulted in antigen specific APC elimination was then examined. Using the generated viruses, expression of the transgenic TCRs in CD8+ T cells was first confirmed using flow cytometric or rtPCR examination of transgene TCR chains and antigen specific killing of splenocytes treated with LPS to enhance expression of MHC class II molecules and antigen presentation was examined (FIG. 2; Table 1).

Table 1 shows the specific lysis of peptide-pulsed splenocytes by the engineered CD8+ T cells. LPS treated (MHC class II increase whole splenocytes were used as lytic target following 1 hour culture with each concentration of cognate MOG or OVA peptide. Cells were cultured for 3 days with the engineered CD8+ T cells listed (OT-II TCR, 2D2 TCR or B8 TCR). Remaining cell numbers were compared to an internal control population that was not loaded with peptide.

TABLE 1

| Cytotoxicity | OT-II TCR | 2D2 TCR | B8 TCR |
|---|---|---|---|
| Splenocytes with | 1 ± 1 | 5 ± 3 | 15 ± 6* |
| 20 uM MOG | | | |
| peptide | | | |
| Splenocytes with | 4 ± 3 | 17 ± 3* | 27 ± 5* |
| 200 uM MOG | | | |
| peptide | | | |
| Splenocytes with 5 | 32 ± 7* | 3 ± 1 | 2 ± 2 |
| uM OVA peptide | | | |

*= P <0.05

All three TCRs showed statistically significant peptide specific cytotoxic capacity against their expected cognate antigen with no observed off target cytotoxic effects. It was thus found that transduction of with CD8+ T cells granted antigen specific killing capacity. Notably, the concentrations of peptide necessary to trigger cytotoxicity of targets was substantially different between TCRs, with the 2D2 in particular requiring significantly greater concentrations than the B8 and OT-II TCRs. Examination of killing capacity over three different preparations of transduced cells revealed 17-27% antigen specific killing directed to the highest concentrations of pulsed MOG peptide. Similar levels of killing were seen with ovalbumin peptide pulsed targets when the ovalbumin specific OT-II TCR was transduced into CD8 cells demonstrating that killing is not restricted to TCRs against MOG antigens. Instead these lines apparently acquired antigen specificity for the TCR transgene while maintaining previous effector capacities, findings that fit with previous demonstrations of functional MHC class II restricted CD8+ T cells in the context of CD4 genetic deficiency (Hansen et al. (2013) Science 340: 1237874; Pearce et al. (2004) J Immunol 173: 2494-9) and the limited signaling required for cytotoxicity induction (Holler et al. (2003) Immunity 18: 255-64).

Having determined the capacity of these cells to recognize antigen in vitro, capacity of the cells to initiate lysis of targets in vivo was assessed. To determine in vivo cytotoxic capacity, 5×10⁶ CHASE cells were transferred to naïve mice. After 3 days, hosts received MOG and OVA peptide-pulsed LPS-activated CD45.1 congenic splenocytes by intravenous injection, at a 1:1 ratio. To track transferred cells, both the CD45.1 marker and 20-fold different concentrations of CF SE were used in the MOG versus OVA peptide-pulsed cells. Relative survival of the two peptide pulsed groups could then be determined within the spleen after 48 hours (Table 2). As shown in Table 2, CHASE cells demonstrated antigen specific lysis of adoptively transferred antigen presenting cells with 48 hours of transfer.

TABLE 2

| | | | | No |
|---|---|---|---|---|
| Cytotoxicity | OT-II | B8 | 2D2 | CHASE |
| Ratio of | 6.2 ± 2.5 | 0.4 ± 0.1 | 0.7 ± 0.3 | 0.9 ± 0.1 |
| MOG/OVA | | | | |
| pulsed APC | | | | |

Interestingly, there were significant differences in the capacity of the B8 and 2D2 TCR engineered CD8+ cells to lyse cells presenting different concentrations of MOG peptide. These striking differences in functional differences may be associated with the relative affinity of each TCR for MOG antigen.

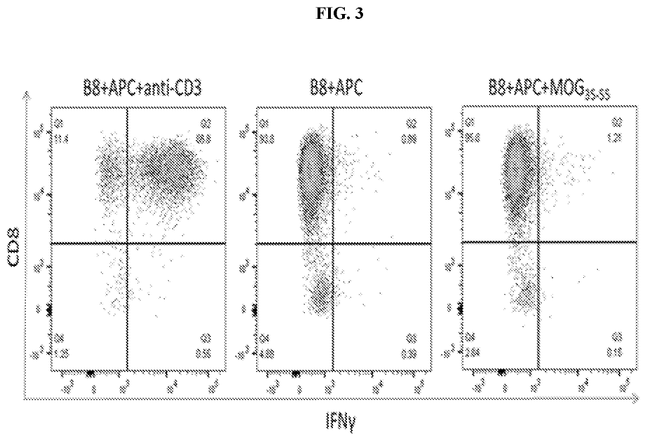

Having demonstrated antigen-specific lysis both in vitro and in vivo, the ability of CHASE T cells to exhibit typical cytokine responses to antigen were analyzed. Surprisingly, all three types of CHASE cells had deficient cytokine responses to antigen even at concentrations that were sufficient to drive cytotoxicity (data for B8 cells shown in FIG. 3). B8 engineered CD8+ T cells were incubated with or without a 50 μM concentration of the peptide recognized by the B8 TCR for 16 hours and then examined for production of interferon gamma (FIG. 3), IL-17, IL-2, GM-CSF, or TNF alpha. No significant expression of cytokine was shown following stimulation. This was not associated with a general inability of the cell to produce cytokine as treatment of the cells with either CD3 or PMA and ionomycin was sufficient to produce large amounts of interferon gamma in the majority of cells (FIG. 3). Thus, the data suggests that the defect in cytokine production is not associated with a simple switch in cytokine usage, a finding supported by the capacity of all CHASE cells to produce copious amounts of interferon gamma following anti-CD3 stimulation (FIG. 3).

The inability to drive cytokine responses was observed in all three lines over a range of peptide concentrations presented by either irradiated splenocytes (shown in FIG. 3) or bone marrow derived dendritic cells (data not shown). The deficit observed was surprising given the studies in CD4 deficient mice that showed MHC class II-restricted killing and cytokine production (Pearce et al. (2004) J Immunol 173: 2494-9), but fits with data in mature CD4+ T cells that demonstrated that co-receptor blockade could abrogate cytokine production in response to antigen stimulation (Madrenas et al. (1997) J Exp. Med.: 185: 219-29).

The surprising deficit in cytokine production in the engineered CD8+ cells may be ideal for cytotoxic manipulation of autoimmunity as it allows for cytotoxicity in the absence of the powerful inflammatory effects T cell produced cytokines can have on neuroinflammation. While not being bound by theory, this deficiency in cytokine production may be associated with the lack of CD4+ co-receptor activity in the engineered CD8+ cells during cognate antigen interactions.

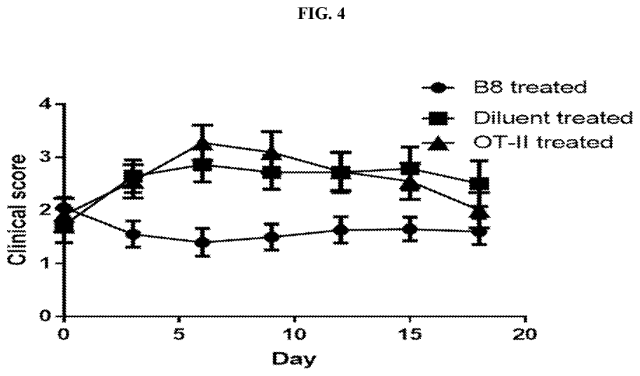

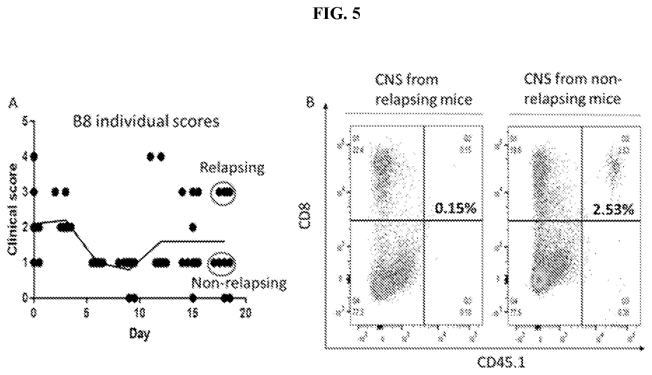

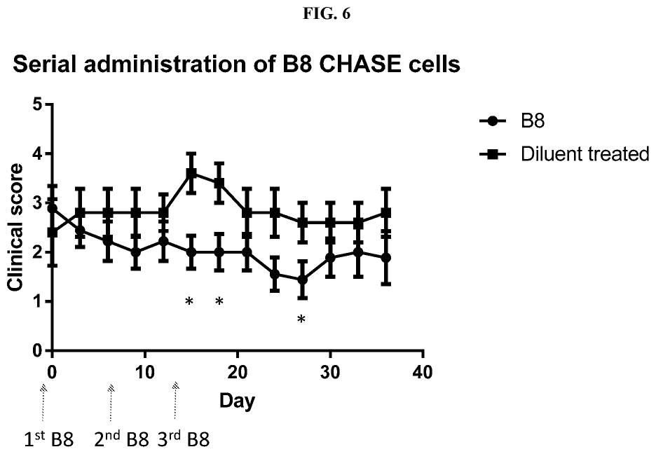

The ability of the engineered CD8+ cells to ameliorate demyelinating autoimmunity in experimental autoimmune encephalomyelitis (EAE) was then examined. B8 and OT-II TCR CHASE cells were used in this experiment as they had demonstrated significantly greater in vitro responsiveness to antigen than the 2D2 CHASE cells. Two days after the onset of EAE symptoms, 5×10⁶ cells were injected into the mice. It was found that transfer of MOG responsive B8 cells significantly decreased clinical scores of EAE afflicted mice within 3 days of adoptive transfer. This clinical benefit appeared to be antigen specific as the effect was observed in EAE mice following transfer of equal numbers of OT-II CHASE cells. (FIG. 4). B8 CD8+ T cell-treated mice were examined at day 17 following transfer to examine distribution of injected B8 CD8+ T cells within the spleen and CNS. Interestingly a subset of B8 CD8+ T cell treated mice that maintained the amelioration of disease for at least 17 days following transfer had a significant population of transferred cells within both the spleen and CNS (FIG. 5). The relative frequency of the engineered CD8+ T cells was increased within the CNS compared to the spleen suggesting preferential trafficking/retention of engineered CD8+ T cells within the target organ. In contrast, mice showing a disease relapse had very few engineered CD8+ T cells within the CNS (FIG. 5) or the spleen suggesting that engineered CD8+ T cells are relatively quickly lost from both the periphery and target organ in those mice. Similar results were observed for serial administration of the B8 engineered CD8+ T cells (FIG. 6).

These data demonstrate that the underlying concept of the engineered CD8+ T cell approach is sound as adoptive transfer of myelin specific engineered CD8+ T cells can ameliorate established neuroinflammatory disease.

### Example 2

In this example, the capacity of engineered CD8+ T cells specific for autoantigen-MHC II to ameliorate neuroinflammation in different models of EAE will be determined. We will utilize multiple EAE models to determine the capacity of engineered CD8+ T cells to either block development or treat established EAE neuroinflammation. Use of different systems will allow us to confirm the therapeutic potential of these cells in both B-cell dependent and independent disease and the capacity of cells to block adoptively transferred disease allowing us to determine the effects of engineered CD8+ T cell treatment in the absence of the large amounts of exogenous myelin antigens used to generate active disease. We will further extend our preliminary findings in the MOG₃₅₋₅₅ peptide induction model to determine if introduction of engineered CD8+ T cells at peak disease can still impact disease. Together these systems will allow us to confirm that mature CD8+ T cells capable of recognizing and killing MHC class II/auto-antigen complex decorated antigen presenting cells will reduce CD4+ T cell driven autoimmune neuroinflammation under many different neuroinflammatory conditions.

We will use three different protocols for EAE disease induction; specifically, we will extend our studies in an active immunization with the MOG₃₅₋₅₅ immunodominant peptide (B cell independent disease), active immunization with full length MOG protein (B cell dependent disease), and adoptive transfer of MOG₃₅₋₅₅ specific CD4 T cells recovered from actively immunized mice. It is expected that the engineered CD8+ T cell treatment will ameliorate EAE.

Cytometrically sorted congenic (CD45.1 or CD90.1+C57BL/6 mice) murine CD8 T cells will be activated using anti-CD3 and anti-CD28 stimulation and transduced with vectors for production of B8 and OT-II engineered CD8+ T cells. Some groups will additionally receive vectors that drive expression of the full-length mouse CD4. Resultant transduced cells will be sorted based on GFP and CD4 expression. An aliquot of cells will be examined for transgenic TCR and CD4 prevalence to determine efficacy of transgenic expression. Cells will then be expanded in vitro, and validated for MHC class II recognition of syngeneic APC loaded with MOG₃₅₋₅₅ by examination of in vitro cytotoxicity against MOG-pulsed LPS activated spleen cells to determine functional efficacy. Cells will also be examined for expression of interferon gamma, IL-2, IL-17 and GM-CSF. These cells will then be transferred to MOG₃₅-55/CFA/Pertussis toxin injected C57BL/6 mice 2 days after the first appearance of EAE symptoms, as done in our preliminary data (FIGS. 4 and 5). Mice will be followed for disease severity, and weight, for 30 days. Mice will then be euthanized and either examined for CNS cell constituents by flow cytometry, or tissue will be frozen for histological examination. We will use conventional and confocal microscopy to determine histological markers of disease and T cell localization within different sections of the CNS as described previously (McCandless et al. (2009) J Immunol 183: 613-20). All experiments will utilize CNS tissues from non-manipulated mice as negative controls and will be graded in a blinded fashion. Sections will be stained for congenic markers, VCAM-1, and nuclei counterstained with ToPro3 to determine lesion formation. Gross lesion numbers and pathological severity will be determined for each slide using CD90.1 and VCAM-1 expression, respectively. Additionally, we will examine sections for engineered CD8+ T cells congenic markers as well as GFP expression to determine adoptively transferred cell localization, as well as continued expression of the engineered CD8+ T cells transgenic TCR. At least ten serial sections/group will be examined to insure reproducibility. We expect that expression of CD4 will result in a correction of the cytokine production defect we have observed in engineered CD8+ T cells. We expect that this cytokine production will also impact the therapeutic effect of engineered CD8+ T cell transfer in the previously studied model by preventing amelioration of clinical scores.

The impact of multiple engineered CD8+ T cell injections on amelioration of established EAE disease will also be studied. In this experiment, the engineered CD8+ T cells will be transferred to MOG35-55/CFA/Pertussis toxin injected C57BL/6 mice with fulminant signs of EAE disease, as measured by appearance of clinical signs at least 2 days prior to adoptive transfer as was done in our preliminary studies. Mice will then receive new injections of the appropriate engineered CD8+ T cell every 7 days for a total of three injections over 21 days. Mice will be followed for disease severity, and weight, for a total of 40 days. Mice will then be euthanized and either examined for CNS cell constituents by flow cytometry, or tissue will be frozen for histological examination. All experiments will utilize CNS tissues from non-manipulated mice as negative controls and will be graded in a blinded fashion and histological studies will be performed. We expect that the multiple injections treatment protocol will result in a continued amelioration of clinical scores throughout at least the 21 days of continued transfer.

The amelioration of disease following establishment of peak EAE symptoms will also be studied. In this experiment, engineered CD8+ T cells will be transferred to MOG35-55/CFA/Pertussis toxin injected C57BL/6 mice with fulminant signs of EAE disease, as measured by appearance of clinical signs at least 7 days prior to adoptive transfer or 1 week after peak disease (as measured by maintenance or decrease of clinical score for at least 1 week). Mice will be followed for disease severity, and weight, for 30 days. Mice will then be euthanized and either examined for CNS cell constituents by flow cytometry, or tissue will be frozen for histological examination. All experiments will utilize CNS tissues from non-manipulated mice as negative controls and will be graded in a blinded fashion and histological studies will be performed. We expect that treatment beginning at peak disease will still result in transient amelioration of clinical scores.

Modulation of a B cell dependent model of EAE by treatment with the engineered CD8+ T cells will also be examined. In this experiment, the engineered CD8+ T cells will be transferred to MOG₁₋₁₂₅/CFA/Pertussis toxin injected C57BL/6 mice with fulminant signs of EAE disease, as measured by appearance of clinical signs at least 2 days prior to adoptive transfer. Mice will be followed for disease severity, and weight, for 30 days. Mice will then be euthanized and either examined for CNS cell constituents by flow cytometry, or tissue will be frozen for histological examination. All experiments will utilize CNS tissues from non-manipulated mice as negative controls and will be graded in a blinded fashion and histological studies will be performed. We expect that treatment with the engineered CD8+ T cells will have a therapeutic effect on this model due to the sensitivity of antigen specific B-cells to CD8 mediated lysis.

Modulation of an adoptive transfer model of EAE by treatment with the engineered CD8+ T cells will also be examined. In this experiment, the engineered CD8+ T cells will be transferred to C57BL/6 mice that had previously been injected with MOG35-55 specific activated polyclonal CD4+ T cells with fulminant signs of EAE disease, as measured by appearance of clinical signs at least 2 days prior to adoptive transfer or in to mice 1 week after peak (as measured by maintenance or decrease of clinical score for at least 1 week). Mice will be examined for CNS cell constituents by flow cytometry, or tissue will be frozen for histological examination. All experiments will utilize CNS tissues from non-manipulated mice as negative controls and will be graded in a blinded fashion. All experiments will utilize CNS tissues from non-manipulated mice as negative controls and will be graded in a blinded fashion and histological studies will be performed. We expect that the treatment with the engineered CD8+ T cells will have a therapeutic impact on passive EAE as the animals will not have high concentrations of exogenous antigen in the periphery as occurs in active immunization EAE models.

### Example 3

In this example, we will determine the effect the engineered CD8+ T cells have on distinct WWII expressing CNS cell populations in the context of EAE. We will examine MHC class II positive CNS cell populations to determine if administration of the engineered CD8+ T cells has gross effects on cell numbers or relative abundance. We will also use a modified direct ex vivo antigen detection assay (Nandi et al. (2009) Infection and immunity 77: 4643-53) to determine if MOG₃₅₋₅₅ antigen presentation in CNS resident leukocytes is modified by treatment with the engineered CD8+ cells. This experiment will establish that transfer of these MHC class II/myelin antigen responsive cytotoxic CD8+ T cells results in the recognition and elimination of myelin presenting antigen presenting cells (APC).

We will examine both in vitro generated and cell sorted T cells to establish that engineered CD8+ T cells will be sufficient to lyse antigen coated APC in vivo. This series of experiments will determine what specific subsets of APC, if any, are differentially impacted by treatment with the engineered CD8+ T cells. We will utilize our CD8+ T cells and an adoptive transfer system to determine what effect engineered CD8+ T cell antigen recognition will have on inflammation associated APC cell populations.

In vivo targeting of exogenous dendritic cells and B cells will be examined. Flow cytometrically sorted murine CD8 T cells will be activated using anti-CD3 and anti-CD28 stimulation and transduced with this vector. Resultant transduced cells will be sorted, expanded in vitro, and validated for MHC class II recognition of syngeneic APC loaded with MOG₃₅₋₅₅ by examination of in vitro cytotoxicity against MOG-pulsed LPS activated spleen cells. To determine in vivo cytotoxic capacity and titrate doses of engineered CD8+ T cells we will transfer engineered CD8+ T cells at varying doses. Hosts will then receive MOG₃₅₋₅₅ peptide pulsed bone marrow-derived dendritic cells (BMDC) and non-pulsed BMDC by IV injection, at a 1:1 ratio, using either congenic (CD45.1 or CD90.1) differences or differential staining with a cell incorporating florescent dye. We will then determine relative survival of the two DC groups within the spleen after 48 hours. Similar experiments will be performed with LPS activated B cell populations. We expect that administration of some dose of the engineered CD8+ cells will result in in vivo elimination of exogenously derived, peptide coated APC. Because engineered CD8+ T cells will be tested for in vitro cytotoxicity prior to usage in these experiments any difference in in vivo versus in vitro cytotoxicity would allow us to interrogate changes associated with in vivo APC residence that impact sensitivity to cytotoxicity.

The impact of treatment with the engineered CD8+ T cells on CNS APC populations will be examined. Flow cytometrically sorted engineered CD8+ T cells will be produced as outlined above. To determine in vivo effects of engineered CD8+ T cells on specific APC populations within the CNS we will transfer engineered CD8+ T cells at varying doses into untreated or EAE diseased mice. The presence of transferred cells within the CNS will be followed over time to determine if engineered CD8+ T cells preferentially populate the CNS in inflamed vs normal conditions. CD45+ MHC class II+ cell populations will then be examined using flow cytometric methods adopted from a previously published protocol (Wlodarczyk, et al. (2014) J Neuroinflammation 11: 57) to determine cell population absolute numbers and relative abundance, and document any changes associated with administration of the engineered CD8+ T cells. We expect that administration of the engineered CD8+ T cells will have a specific impact a particular CNS APC subset. If antigen presentation of myelin epitopes is very different between APC we would expect there to be a substantive change associated with administration of the cells.

### Example 4

In this example, MHC class II/self-antigen elimination mediated by engineered CD8+ cells as a method for limiting the autoimmunity involved in Type I diabetes will be examined. Activity of autologous CD8+ T cells transduced with a previously characterized TCR that recognizes the influenza hemagglutinin antigen (HA) in the context of the I-A^{d} MHC class II molecule (TCR-HA) will used to establish targeting and elimination of HA-expressing APCs. The engineered CD8+ cells will be tested for their capacity to specifically lyse HA loaded antigen presenting cells, and utilize adoptive transfer of these cells into rat insulin promotor driven HA expressing (RIP-HA) mice to observe the capacity of the engineered CD8+ cells to either block or reverse diabetes induction.

The capacity of engineered CD8+ cells to block development and/or ameliorate established symptoms of diabetes will be determined. We will utilize the RIP-HA model of diabetes induction to determine the capacity of CD8+ cells to either block development of disease or treat established islet inflammation.

The data described in Example 1 above revealed that CD8+ T cells transduced with TCRs known to recognize MHC class II/peptide complexes are capable of specifically killing cells bearing those complexes. Further in vivo studies supported that transfer of these MHC class II/myelin antigen responsive cytotoxic CD8+ T cells can result in amelioration of established neuroinflammatory disease suggesting that a similar approach will impact diabetes.

For examination of diabetes prevention, cytometrically sorted congenic (CD45.1 or CD90.1+C57BL/6 mice) murine CD8 T cells will be activated using anti-CD3 and anti-CD28 stimulation and transduced with vectors for production of HA and OT-II engineered CD8+ cells. An aliquot of cells will be examined for transgenic TCR and CD4 prevalence to determine efficacy of transgenic expression. Cells will then be expanded in vitro, and validated for MHC class II recognition of syngeneic APC loaded with HA₁₁₀₋₁₂₀ peptide by examination of in vitro cytotoxicity against HA peptide pulsed LPS activated spleen cells to determine functional efficacy. Cells will also be examined for expression of interferon gamma, IL-2, IL-17 and GM-CSF. These cells will then be transferred to RIP-HA mice 2 days before adoptive transfer of diabetogenic purified CD4+ TCR-HA expressing cells. Mice will be followed for blood glucose levels for 30 days. Mice will then be euthanized and pancreatic tissue will be frozen for histological examination. We will use conventional and confocal microscopy to determine histological markers of disease and T cell localization within pancreatic islets as described previously. All experiments will utilize pancreatic tissues from non-manipulated mice as negative controls and will be graded in a blinded fashion. Sections will be stained for congenic markers and nuclei counterstained with ToPro3 to determine lesion formation. At least ten serial sections/group will be examined to insure reproducibility.

For examination of diabetes amelioration, TCR-HA engineered CD8+ cells will be introduced to the RIP-HA mice immediately after CD4+ T cell induction of diabetes has occurred in the RIP-HA mice as measured by a blood glucose level >200 mg/dl. Mice will be followed for blood glucose levels and weight loss for 10 days, then euthanized for pancreatic tissue examination. It is expected that administration of the engineered CD8+ cells will block development and/or ameliorate established symptoms of diabetes.

All patents, patent applications, provisional applications, and publications referred to or cited herein are incorporated by reference in their entirety, including all figures and tables, to the extent they are not inconsistent with the explicit teachings of this specification.

What is claimed is:

1. An engineered CD8+ T cell comprising a heterologous nucleic acid encoding a T cell receptor (TCR), wherein the TCR binds to a self-antigen bound to a major histocompatibility complex (MHC) class II.

2. The engineered CD8+ T cell of claim 1, wherein the engineered CD8+ T cell binds to a cell that expresses the self-antigen bound to MHC class II.

3. The engineered CD8+ T cell of claim 1, wherein the engineered CD8+ T cell is able to lyse a cell that expresses the self-antigen bound to MHC class II.

4. The engineered CD8+ T cell of claim 2 or claim 3, wherein the cell that expresses the self-antigen bound to MHC class II is a dendritic cell, a macrophage, a monocyte, a microglial cell, or an astrocyte.

5. The engineered CD8+ T cell of any one of claims 1-4, wherein the MHC class II comprises H-2A, HLA-DP, HLA-DM, HLA-DOA, HLA-DOB, HLA-DQ, or HLA-DR.

6. The engineered CD8+ T cell of any one of claims 1-4, wherein the MHC class II comprises HLA-DR2, HLA-DR3, HLA-DR4, HLA-DR11, HLA-DR15 or HLA-DQ6.

7. The engineered CD8+ T cell of any one of claims 1-6, wherein the engineered CD8+ T cell decreases antigen specific activation of CD4+ cells when administered to a subject having an autoimmune disease.

8. The engineered CD8+ T cell of claim 7, wherein the autoimmune disease is a neuroinflammatory disease.

9. The engineered CD8+ T cell of claim 7, wherein the autoimmune disease is multiple sclerosis, diabetes, rheumatoid arthritis, myasthenia gravis, psoriasis, systemic lupus erythematosus, autoimmune thyroiditis, Graves' disease, inflammatory bowel disease, autoimmune uveoretinitis, myocarditis, and polymyositis.

10. The engineered CD8+ T cell of any one of claims 1-9, wherein the self-antigen is a central nervous system (CNS) antigen.

11. The engineered CD8+ T cell of any one of claims 1-9, wherein the self-antigen is selected from myelin oligodendrocyte glycoprotein (MOG), myelin basic protein (MBP), myelin associated glycoprotein (MAG), and proteolipid protein (PLP).

12. The engineered CD8+ T cell of any one of claims 1-9, wherein the self-antigen is MOG₃₅₋₅₅.

13. The engineered CD8+ T cell of any one of claims 1-9, wherein the self-antigen is a diabetes mellitus associated antigen, a rheumatoid arthritis associated antigen, myocarditis associated self-antigen, or a thyroiditis associated antigen.

14. The engineered CD8+ T cell of claim 13, wherein the self-antigen is insulin, chromogranin A, glutamic acid decarboxylase 1 (GAD67), glutamic acid decarboxylase 2 (GAD65) or islet-specific glucose-6-phosphatase catalytic subunit-related protein.

15. The engineered CD8+ T cell of any one of claims 1-14, wherein the TCR is derived from a CD4+ T cell.

16. The engineered CD8+ T cell of any one of claims 1-14, wherein the TCR is 2D2, B8, or bdc2.5.

17. The engineered CD8+ T cell of any one of claims 1-16, wherein the TCR is a chimeric antigen receptor (CAR) comprising (i) an extracellular antigen binding domain; (ii) a transmembrane domain; and (iii) an intracellular domain.

18. The engineered CD8+ T cell of claim 17, wherein the extracellular antigen binding domain binds to the self-antigen bound to MHC Class II.

19. The engineered CD8+ T cell of any one of claims 17-18, wherein the extracellular antigen binding domain is derived from an antigen-binding portion of an antibody, a T cell receptor, or a B-cell receptor.

20. The engineered CD8+ T cell of claim 19, wherein the T cell receptor is 2D2, B8 or bdc2.5.

21. The engineered CD8+ T cell of any one of claims 17-20, wherein the extracellular antigen binding domain comprises a single chain variable fragment (scFV).

22. The engineered CD8+ T cell of claim 21, wherein the extracellular antigen binding domain comprises an scFv of 2D2 or B8.

23. The engineered CD8+ T cell of any one of claims 17-22, wherein the intracellular domain comprises one or more costimulatory domains.

24. The engineered CD8+ T cell of claim 23, wherein the one or more costimulatory domains are selected from a CD28 costimulatory domain, a CD3ζ-chain, a 4-1BBL costimulatory domain, or any combination thereof.

25. The engineered CD8+ T cell of any one of claims 1-24, wherein the nucleic acid encoding the TCR is operably linked to an inducible promoter or a conditional promoter.

26. A method for treating an autoimmune disease or condition, comprising administering an engineered CD8+ T cell of any one of claims 1-25 to a subject in need thereof.

27. The method of claim 26, wherein the autoimmune disease or condition is multiple sclerosis.

28. The method of claim 26, wherein the autoimmune disease or condition is diabetes.

29. The method of any one of claims 26-28, wherein administering the engineered CD8+ T cell decreases antigen specific activation of CD4+ cells in the subject, decreases tissue damage in the subject, and/or decreases autoimmune inflammation in the subject compared to no administration of the engineered CD8+ T cell.

30. The method of any one of claims 26-29, further comprising administering one or more additional therapeutic agents.

31. The method of claim 30, wherein the one or more additional therapeutic agents is an anti-inflammatory agent or an immunosuppressive agent.

32. The method of any one of claims 26-31, wherein the CD8+ T cells are derived from an autologous donor or an allogenic donor.

33. The method of any one of claims 26-32, wherein the subject is a human subject.

34. A method for preparing an engineered CD8+ T cell, comprising transducing cytotoxic CD8+ T cells with a nucleic acid encoding a T cell receptor that binds to a self-antigen bound to a major histocompatibility complex (MEW) class II.

35. A method for preparing an engineered CD8+ T cell, comprising transducing cytotoxic CD8+ T cells with a nucleic acid encoding a T cell receptor from isolated CD4+ T cells or a chimeric antigen receptor comprising an antigen binding domain thereof, wherein CD4+ T cells the bind to a self-antigen from a subject having an autoimmune disease.

36. A method for preparing an engineered T cell comprising:
(a) isolating CD4+ T cells that bind to a self-antigen from a subject having an autoimmune disease;
(b) isolating nucleic acid encoding a T cell receptor from the isolated CD4+ T cells; and
(c) transducing cytotoxic CD8+ cells with nucleic acid encoding the T cell receptor from the isolated CD4+ T cells or a chimeric antigen receptor comprising an antigen binding domain thereof.

37. The method of claim 36, wherein the CD4+ T cells are MHC Class II-restricted T cells.

38. The method of any one of claims 34-37, further comprising expanding the transduced CD8+ cells.

39. The method of claim 38, wherein expanding the transduced CD8+ cells comprises stimulation with and anti-CD3 and/or and anti-CD28 antibody.

40. The method of any one of claims 34-39, further comprising administering the transduced CD8+ cells to a subject in need thereof.

41. The method of claim 40, wherein the subject has an autoimmune disease or condition.

42. The method of claim 41, wherein the autoimmune disease is multiple sclerosis or diabetes.

43. An engineered CD8+ T cell according to any one of claims 1-25, for treating an autoimmune disease in a subject in need thereof.

44. Use of an engineered CD8+ T cells according to any one of claims 1-25, in the preparation of a medicament for treating an autoimmune disease.
